(19) United States
(12) Patent Application Publication
(10) Pub. No.: US 2025/0002951 A1
(43) Pub. Date: Jan. 2, 2025

(54) SYSTEMS AND METHODS FOR THE PRODUCTION OF BIOGASES FROM A LIGNOCELLULOSIC FEEDSTOCK

(71) Applicant: THE PENN STATE RESEARCH FOUNDATION, University Park, PA (US)

(72) Inventors: John Michael REGAN, University Park, PA (US); Katharine HIRL, University Park, PA (US); Michael John SHREVE, University Park, PA (US); Anahita BHARADWAJ, University Park, PA (US); Thomas RICHARD, University Park, PA (US)

(21) Appl. No.: 18/709,342

(22) PCT Filed: Nov. 10, 2022
(86) PCT No.: PCT/US2022/409582

§ 371 Date: May 10, 2024

Related U.S. Application Data
(60) Provisional application No. 63/277,962, filed on Nov. 10, 2021.

(51) Int. Cl. C12P 7/6409; C12P 5/02

(52) U.S. Cl. CPC C12P 7/6409; C12P 5/023

(57) ABSTRACT

Disclosed are systems and methods for the production of a biogas from a lignocellulosic biomass. These methods can include inoculating a feedstock mixture with a mixed microbial community; using a pH adjusting agent to increase a pH of the feedstock mixture to an alkaline pH; incubating the feedstock mixture at a thermophilic temperature; contacting the digestate with a pH adjusting agent to a substantially neutral pH; and incubating the first digestate to form a second biogas. Also provided are systems to convert a lignocellulosic feedstock to biogas having a first reactor operating at an alkaline pH and a thermophilic temperature; and a second reactor operating at a substantially neutral pH. The systems and methods disclosed herein can produce substantially pure biogas and a residual biogas stream having high carbohydrate conversion without the addition of non-digestible solids and can also be used to produce volatile fatty acids (VFA).

### CROSS REFERENCE TO RELATED APPLICATIONS

This application claims the benefit of priority to U.S. Provisional Application No. 63/277,962, filed Nov. 10, 2021, which is incorporated by reference herein in its entirety.

### STATEMENT ACKNOWLEDGING GOVERNMENT SUPPORT

This invention was made with government support under Grant No. DE-AC05-00OR22725 awarded by the Department of Energy and under Hatch Act Project No. PEN04671 awarded by the United States Department of Agriculture/NIFA. The Government has certain rights in the invention.

### TECHNICAL FIELD

This application relates generally to systems and methodology for forming biogases from carbonaceous feedstocks.

### BACKGROUND

Anaerobic digestion is a technology that was originally used as a waste management strategy for organic waste streams such as sewage sludge, manures, and food wastes to address odors, pathogens, and waste disposal, with renewable energy as a byproduct. In recent years, anaerobic digestion processes have been designed with a primary goal of generating renewable fuels and/or chemicals.

Compared to traditional organic waste streams, lignocellulosic biomass shows promise as a feedstock for renewable fuel generation due to its abundance, added benefits to soil and water health, and provision of ecosystem services. Although lignocellulosic biomass is abundant, the lignin fraction forms a matrix around the cellulose and hemicellulose, providing protection from enzymatic hydrolysis and resulting in a feedstock that is recalcitrant to biological conversion. Thus, this recalcitrance must be overcome to fully realize the potential of lignocellulosic biomass as a feedstock for the production of renewable fuels and chemicals.

### SUMMARY

Provided herein are systems and methods related to the formation of high purity biogas and a residual biogas stream having high carbohydrate conversion from a lignocellulosic feedstock. These systems and methods can address problems associated with the processing of recalcitrant lignocellulosic feedstock without necessarily employing pretreatment steps.

These methods can involve two sequential digestion reactions. In the first reaction, lignocellulosic feedstock can be digested at an alkaline pH and thermophilic temperature, producing a first high purity biogas. Subsequently, the first digestate can be digested at a neutral pH to afford a second biogas. These sequential digestions can be performed in a single reactor (whose conditions are shuttled between the conditions used in the first digestion reaction and the conditions used in the second digestion reaction). Alternatively, the first digestion reaction can be performed in a first reactor at which point the first digestate can be transferred to a second reactor where the second digestion is performed.

Accordingly, also provided are systems that include two reactors: a first reactor, wherein the first reactor is configured to receive the lignocellulosic feedstock and a mixed microbial community; and wherein the first reactor operates at an alkaline pH and a thermophilic temperature to anaerobically digest the lignocellulosic feedstock for a first retention time thereby producing a first biogas and a first digestate; and a second reactor configured to receive the first digestate, wherein the second reactor operates at a substantially neutral pH to anaerobically digest the first digestate for a second retention time thereby producing a second biogas and a second digestate.

In some embodiments, the systems and method produce a first high purity biogas comprising, for example, at least 89% methane by volume, such as at least 90% methane by volume, at least 95% methane by volume, or at least 97% methane by volume. In other embodiments, the carbohydrate conversion of the lignocellulosic biomass, as determined by quantitative saccharification, is at least 30%, such as at least 35%, at least 40%, at least 45%, or at least 50%.

Typically, a high purity of methane (>97% by volume) is necessary for a biogas to be utilized as renewable natural gas. Other methods of anerobic digestion under alkali conditions afford a significantly lower purity. Thus, additional purification steps are required to effectively use these biogases as a fuel source. The system and method disclosed herein reduces this procedural strain by showing a high carbohydrate conversion yielding a substantially pure biogas and a residual biogas stream.

Also disclosed herein are methods for forming a volatile fatty acid (VFA) from a lignocellulosic biomass. The methods can include inoculating a feedstock mixture comprising a lignocellulosic biomass with a mixed microbial community, contacting the feedstock mixture with effective amount of a pH adjusting agent to increase a pH of the feedstock mixture to an alkaline pH, and incubating the feedstock mixture anaerobically for a retention time at a thermophilic temperature of at least 45° C. thereby forming a digestate comprising a VFA. The VFA can then be isolated (if desired) and employed as a chemical feedstock for subsequent downstream chemical processes.

The foregoing and other features and advantages of the invention will become more apparent from the following detailed description, which proceeds with reference to the accompanying figures.

### DESCRIPTION OF DRAWINGS

FIG. 1 depicts box plots summarizing process data of the alkaline digester from Example 1.

FIG. 2 depicts box plots comparing carbohydrate conversions of a system with a 10-day retention time at a thermophilic temperature and different pHs for a first alkaline digester.

FIG. 3 depicts box plots comparing methane production of a system with a 10-day retention time at a thermophilic temperature and different pHs for a first alkaline digester.

FIG. 4 depicts box plots comparing volatile fatty acid production of a system with a 10-day retention time at a thermophilic temperature and different pHs for a first alkaline digester.

FIG. 5 is a correspondence plot showing variance in a data set of an alkaline digester.

FIG. 6 depicts a twelve-reactor system configured to anaerobically digest lignocellulosic biomass.

FIG. 7 depicts a single reactor stage configured to anaerobically digest a lignocellulosic biomass.

FIG. 8 is a block flow diagram showing an embodiment of a two-stage anaerobic digestor system.

FIG. 9 is a block flow diagram depicting a single-stage anaerobic digestor system including feedstock cotreatment and in situ recovery of volatile fatty acids (VFAs).

FIG. 10 is a plot showing concentration profiles of the primary volatile fatty acids formed under the reaction conditions of Example 2.

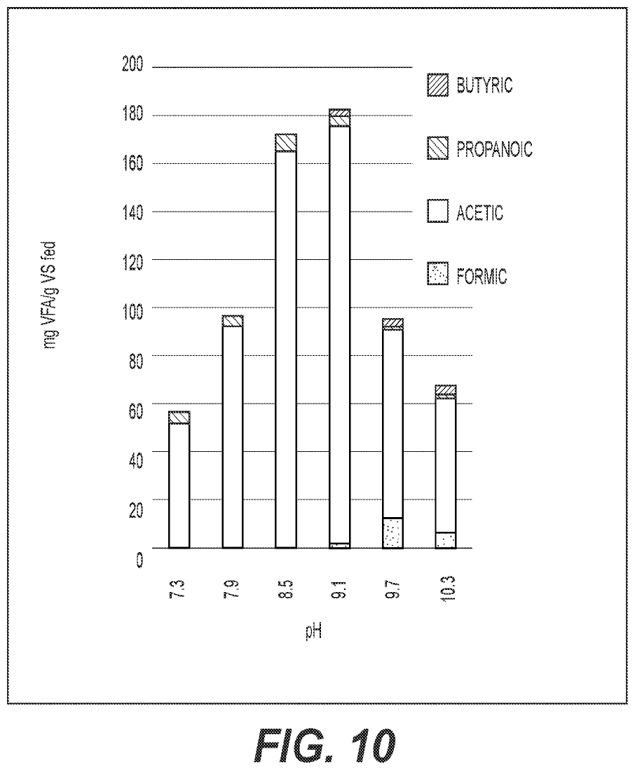

FIG. 11 is a plot depicting volatile fatty acid production rate as a function of pH from Example 2.

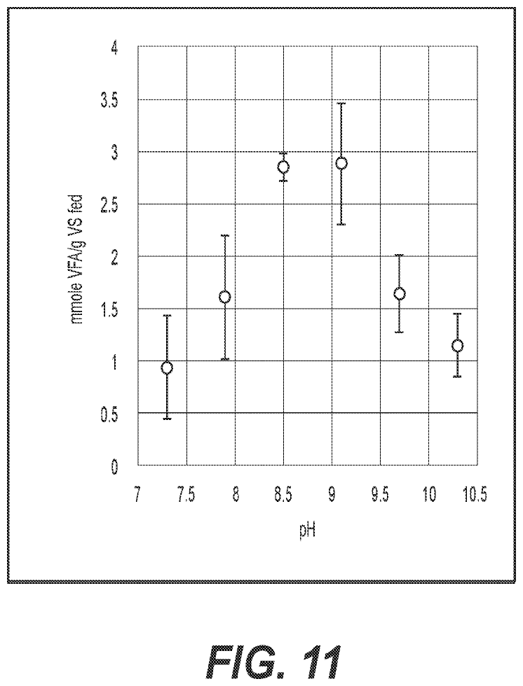

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise.

As used herein, the terms “optional” or “optionally” mean that the subsequently described event or circumstance may or may not occur and that the description includes instances where said event or circumstance occurs and instances where it does not.

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims, which follow, reference will be made to a number of terms that shall be defined herein.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the term “substantially” means that the subsequently described event or circumstance completely occurs or that the subsequently described event or circumstance generally, typically, or approximately occurs.

As used herein, the term “substantially,” in, for example, the context “substantially identical” or “substantially similar” refers to a method or a system, or a component that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the term “primarily”, in, for example, the context of a compound in a group refers to a compound that comprises at least 50% by weigh, at least 55% by weight, at least 60% by weight, at least 65% by weight, at least 70% by weight, at least 75% by weight, at least 80% by weight, at least 85% by weight, at least 90% by weight, or at least 95% by weight.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

As used herein, the terms “first biogas” and “first high purity biogas” are used interchangeably and refer to the biogas stream produced by the first reactor.

As used herein, “thermophilic temperatures” refer to temperatures of at least 45° C., such as at least 50° C., at least 55° C., at least 60° C., or at least 65° C. In some embodiments, thermophilic temperatures can be less than 100° C. (e.g., less than 95° C., less than 90° C., less than 85° C., or less than 80° C.).

As used herein, “solid-liquid” mixture refers to a homogeneous or heterogeneous mixture of one or more solids and one or more liquids, wherein the amount of solids in the mixture is from 0.1% to 75% by weight, such as from 1% to 65% by weight, from 1% to 50% by weight, from 1% to 40% by weight, from 1% to 30% by weight, from 1% to 20% by weight, from 10% to 75% by weight, from 10% to 65% by weight, or from 10% to 50% by weight.

As used herein, the term “soluble pH adjusting agent” refers to any compound capable of adjusting the pH of an aqueous solution, suspension, colloid, emulsion, or other solid-liquid mixture, and that exhibits an aqueous solubility of at least 0.1 g/100 mL at 20° C. (e.g., at least 1.0 g/100 mL at 20° C., at least 5.0 g/100 mL at 20° C., at least 10.0 g/100 mL at 20° C., at least 20.0 g/100 mL at 20° C., at least 25.0 g/100 mL at 20° C., at least 50.0 g/100 mL at 20° C., or at least 75.0 g/100 mL at 20° C.). In some embodiments, substantially all of the soluble pH adjusting agent dissolves in the feedstock mixture at the temperature at which the digestion reaction is performed.

The present invention may be understood more readily by reference to the following detailed description of various aspects of the invention and the examples included therein and to the Figures and their previous and following description.

### Methods of Forming a High Purity Biogas

Disclosed herein are methods for forming a high purity biogas and a residual biogas stream from a lignocellulosic feedstock. These methods can include inoculating a feedstock mixture comprising the lignocellulosic biomass with a mixed microbial community; contacting the feedstock mixture with effective amount of a first pH adjusting agent to increase a pH of the feedstock mixture to an alkaline pH; incubating the feedstock mixture anaerobically for a first retention time at a thermophilic temperature of at least 45° C. thereby forming a first biogas and a first digestate comprising volatile fatty acids; collecting the first biogas; contacting the first digestate with effective amount of a second pH adjusting agent to decrease a pH of the first digestate to a substantially neutral pH; incubating the first digestate anaerobically for a second retention time thereby forming a second biogas; and collecting the second biogas.

In some embodiments the feedstock mixture can be anaerobically incubated at various first and second retention times to produce a high purity gas and a residual biogas stream according to the desired outcome.

For example, in some embodiments, the first retention time can be at least 1 day (e.g., at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, or at least 19 days, at least 20 days, at least 30 days, at least 40 days, at least 50 days, at least 60 days, at least 70 days, at least 80 days, at least 90 days, at least 100 days, at least 150 days, or at least 200 days). In some embodiments, the first retention time can be 20 days or less (e.g., 150 days or less, 100 days or less, 90 days or less, 80 days or less, 70 days or less, 60 days or less, 50 days or less, 40 days or less, 30 days or less, 20 days or less, 19 days or less, 18 days or less, 17 days or less, 16 days or less, 15 days or less, 14 days or less, 13 days or less, 12 days or less, 11 days or less, 10 days or less, 9 days or less, 8 days or less, 7 days or less, 6 days or less, 5 days or less, or 4 days or less).

The first retention time can range from any of the minimum values described above to any of the maximum values described above. For example, the first retention time can be from 1 to 200 days (e.g., from 3 to 200 days, from 3 to 150 days, from 3 to 100 days, from 5 to 100 days, from 10 to 100 days, from 20 to 150 days, from 20 to 100 days, from 30 to 150 days, from 30 to 100 days, from 3 to 15 days, from 3 to 10 days, from 5 to 10 days, or about 10 days).

In some embodiments, the second retention time can be at least 1 day (e.g., at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, or at least 19 days, at least 20 days, at least 30 days, at least 40 days, at least 50 days, at least 60 days, at least 70 days, at least 80 days, at least 90 days, at least 100 days, at least 150 days, or at least 200 days). In some embodiments, the second retention time can be 200 days or less (e.g., 150 days or less, 100 days or less, 90 days or less, 80 days or less, 70 days or less, 60 days or less, 50 days or less, 40 days or less, 30 days or less, 20 days or less, 19 days or less, 18 days or less, 17 days or less, 16 days or less, 15 days or less, 14 days or less, 13 days or less, 12 days or less, 11 days or less, 10 days or less, 9 days or less, 8 days or less, 7 days or less, 6 days or less, 5 days or less, or 4 days or less).

The second retention time can range from any of the minimum values described above to any of the maximum values described above. For example, the second retention time can be from 1 to 200 days (e.g., from 3 to 200 days, from 3 to 150 days, from 3 to 100 days, from 5 to 100 days, from 10 to 100 days, from 20 to 150 days, from 20 to 100 days, from 30 to 150 days, from 30 to 100 days, from 3 to 15 days, from 3 to 10 days, from 5 to 10 days, or about 10 days).

In some embodiments, the feedstock mixture can be contacted with an effective amount of a first pH adjusting agent to increase the pH to an alkaline pH. The use of a pH adjusting agents to increase the pH of the feedstock mixture provides for high conversion of a lignocellulosic biomass without the addition of non-digestible solids to the reaction. Non-digestible solids limit the maximum organic loading rate of the system and thereby reduce the overall efficiency of the digester. In some embodiments, the first pH adjusting agent can comprise a soluble pH adjusting agent.

In some embodiments, the first pH adjusting agent can comprise an organic, or inorganic alkaline material. For example, the first pH adjusting agent may comprise an aqueous base such as sodium bicarbonate, potassium bicarbonate, sodium carbonate, potassium carbonate, sodium hydroxide, potassium hydroxide, calcium carbonate, calcium oxide, calcium hydroxide, magnesium hydroxide, ammonium hydroxide, and dihydroxyaluminum sodium carbonate or any combinations thereof. In certain embodiments, the first pH adjusting agent comprises one or more selected from the group consisting of sodium bicarbonate, potassium bicarbonate, sodium carbonate, potassium carbonate, sodium hydroxide, potassium hydroxide, or any combinations thereof. In certain embodiments, the first pH adjusting agent comprises one or more selected from the group consisting of sodium bicarbonate, potassium bicarbonate, sodium carbonate, potassium carbonate, or any combinations thereof.

The first pH adjusting agent can be present in an amount effective to afford an alkaline pH. In some embodiments, the alkaline pH can be at least 7.5 (e.g., at least 8.0, at least 8.5, at least 9.0, at least 9.5, at least 10.0, at least 10.5, at least 11.0, or at least 11.5). In some embodiments, the alkaline pH can be 12.0 or less (e.g., 11.5 or less, 11.0 or less, 10.5 or less, 10.0 or less, 9.5 or less, 9.0 or less, 8.5 or less, or 8.0 or less).

The alkaline pH can range from any of the minimum values described above to any of the maximum values described above. For example, in some embodiments, the alkaline pH can be from 7.5 to 12.0 (e.g., from 8.0 to 12.0, from 7.5 to 11.0, from 8.0 to 11.0, from 7.5 to 10.0, from 8.0 to 10.0, from 7.5 to 9.5, from 8.0 to 9.5, from 7.5 to 9.0, from 8.0 to 9.0, or from 8.5 to 9.5).

In some embodiments, the feedstock mixture can be buffered so as to maintain an alkaline pH throughout the digestion process. For example, in some embodiments, the feedstock mixture can be buffered so as maintain the pH of the system within 1 pH unit, such as within 0.8 pH units, within 0.6 pH units, within 0.4 pH units, within 0.2 pH units, or within 0.1 pH units throughout the digestion process.

In some embodiments, the first digestate may be contacted with an effective amount of a second pH adjusting agent to decrease a pH of the first digestate to a substantially neutral pH (e.g., a pH of from 6.5 to 7.5, such as a pH of from 6.8 to 7.2, or a pH of about 7). In some embodiments, the second pH adjusting agent can comprise a soluble pH adjusting agent.

In some embodiments, the first pH adjusting agent may comprise an organic or inorganic acid. For example, in some embodiments, the second pH adjusting agent can comprise one or more selected from the group consisting of acetic acid, citric acid, hydrochloric acid, sulfuric acid, nitric acid, oxalic acid, sulfurous acid, carbonic acid, phosphoric acid, tartaric acid, boric acid, formic acid, or any combination thereof.

In some embodiments, the first digestate can be buffered so as to maintain a substantially neutral pH throughout the second digestion process. For example, in some embodiments, the first digestate can be buffered so as maintain the pH of the system within 0.5 pH units, within 0.4 pH units, within 0.2 pH units, or within 0.1 pH units throughout the digestion process.

In some embodiments, the method may be a batch, continuous, or semi-continuous process. In a continuous process the reaction is continuously implemented in an anaerobic digester, adding continuously or semi-continuously the feedstock mixture into the digester; the products of the reaction (the biogas, and the overflow of the digester content) are collected continuously or semi-continuously at one or several outlets of the digester at the rate of the desired advancement for the reaction.

One advantage of this method is its ability to convert recalcitrant lignocellulosic feedstock into methane and carbon dioxide or other desired products (e.g., volatile fatty acids). Lignocellulosic biomass includes plant biomass that is high in cellulose, hemicellulose, and/or lignin. Non-limiting examples include, poplar, oak, eucalyptus, pine, Douglas fir, spruce, wheat straw, barley hull, barley straw, rice straw, rice husks, oat straw, rye straw, corn cobs, corn stalks, sugarcane bagasse, sorghum straw, the whole plant for corn and other grain crops, other grasses, miscanthus, and/or switchgrasses.

The method includes inoculating the feedstock mixture comprising the lignocellulosic biomass with an inoculant. The step of inoculating the feedstock mixture includes any method of depositing, growing, treating, or any other method known in the art to yield a feedstock mixture with an inoculant incorporated therein. In some embodiments, the inoculant comprises a mixed microbial community. The mixed microbial community may comprise, for example, one or more methanogenic microorganisms. In certain embodiments, the mixed microbial community can comprise one or more types of lignocellulosic degrading microorganisms, including, for example, lignocellulosic degrading bacteria and lignocellulosic degrading fungi. In certain embodiments, the mixed microbial community may be obtained from one or more sources of the group consisting of bovine rumen fluid, bovine rumen solids, corn silage, compost, wetland sediment, and/or anaerobic digestate, manures or sludges from farms, wastewater treatment plants or industrial facilities.

In various embodiments, the inoculant includes one or more types of fibrolytic bacteria including, for example, Fibrobacter succinogenes, Ruminococcus flavefaciens, Ruminococcus albus, Butyrivibrio fibrisolvens, Prevotella ruminicola, Eubacterium cellulosolvens, Eubacterium ruminantium, and combinations thereof, and/or one or more rumen fungi such as Piromyces, Neocallimastix, Orpinomyces, Ruminomyces, and combinations thereof.

In various embodiments, the inoculant (e.g., the mixed microbial community) includes one or more genetically modified microorganism(s) such as those disclosed in U.S. Pat. No. 10,662,456. As used herein, a “genetically modified microorganism” and the like refers to the direct human manipulation of a nucleic acid using modern DNA technology. For example, genetic manipulation can involve the introduction of exogenous nucleic acids into an organism or altering or modifying an endogenous nucleic acid sequence present in the organism. For example, a genetic modification can be insertion of a nucleotide sequence into the genome of a microorganism. A genetic modification can also be a deletion or disruption of a polynucleotide that encodes or regulates production of an endogenous or exogenous gene. A genetic modification can also result in the mutation of a nucleic acid or polypeptide sequence. For example, the inoculant can include a microorganism genetically modified to express or overexpress a polypeptide such as cellulase, endoglucanase, cellobiohydrolase, beta-glucosidase. In some embodiments, the inoculant includes one or more microorganisms that are engineered to be tolerant to environmental conditions of the bioreactor (e.g., pH, temperature, concentration of a toxin).

In some embodiments, the inoculant includes a genetically modified microorganism made to increase and/or decrease the cellular production of certain fermentation product(s) such as acetate, acetoin, acetone, acrylic, malate, fatty acid ethyl esters, isoprenoids, glycerol, ethylene glycol, ethylene, propylene, butylene, isobutylene, ethyl acetate, vinyl acetate, other acetates, 1,4-butanediol, 2,3-butanediol, butanol, isobutanol, sec-butanol, butyrate, isobutyrate, 2-OH-isobutryate, 3-OHbutyrate, ethanol, isopropanol, D-lactate, L-lactate, pyruvate, itaconate, levulinate, glucarate, glutarate, caprolactam, adipic acid, propanol, isopropanol, fusel alcohols, and 1,2-propanediol, 1,3-propanediol, formate, fumaric acid, propionic acid, succinic acid, valeric acid, maleic acid, methane, methanol, and poly-hydroxybutyrate. In some embodiments, the inoculant includes a genetically modified microorganism made to increase the production of methane.

The method can be performed under anaerobic conditions. As used herein, the term “anaerobic conditions” is intended to broadly include both anaerobic and microaerophilic environments. Said anaerobic conditions can include oxygen (O₂) levels of 1% or less (e.g., 0.1% or less, 0.01% or less, or 0.001% or less) by volume of O₂ in the gas phase of the environment. Such conditions can be achieved by any method known in the art. One convenient method for achieving effective anaerobic conditions is to add an oxygen scavenging material (e.g., a reducing agent), such as sulfide ion (e.g., as Na₂S), to the feedstock mixture to reduce any oxygen dissolved in the medium. Another method is to house a large volume of material in a closed reactor or vessel or an underground pit or aboveground structure or cavern and let the biological culture consume the residual oxygen. The inoculant can also include nutrients to maintain a suitable biochemical environment including macronutrients such as carbon, nitrogen, phosphorus, potassium, sodium, sulfur, calcium and magnesium, and micronutrients such as iron, nickel, molybdenum, cobalt, tungsten, zinc and selenium. In some embodiments, the nutrients are externally supplemented to the reactant mixtures.

In some embodiments, the feedstock mixture is anaerobically incubated at a thermophilic temperature of at least 45° C. (e.g., at least 50° C., at least 55° C., at least 60° C., at least 65° C., at least 70° C., at least 75° C., at least 80° C., or at least 85° C.). In some embodiments, the feedstock mixture is anaerobically incubated at a thermophilic temperature of 90° C. (e.g., 85° C. or less, 80° C. or less, 75° C. or less, 70° C. or less, 65° C. or less, 60° C. or less, or 55° C. or less).

The feedstock mixture can be anaerobically incubated at a thermophilic temperature ranging from any of the minimum values described above to any of the maximum values described above. For example, in some embodiments, the feedstock mixture can be anaerobically incubated at a thermophilic temperature of from 45° C. to 90° C., such as from 55° C. to 80° C., from 55° C. to 75° C., from 55° C. to 70° C., from 55° C. to 65° C., or from 55° C. to 60° C.

In some embodiments, the first digestate is anaerobically incubated at a temperature of at least 25° C. (e.g., at least 35° C., at least 45° C., at least 50° C., at least 55° C., at least 60° C., at least 65° C., at least 70° C., at least 75° C., at least 80° C., or at least 85° C.). In some embodiments, the first digestate is anaerobically incubated at a temperature of 90° C. or less (e.g., 85° C. or less, 80° C. or less, 75° C. or less, 70° C. or less, 65° C. or less, 60° C. or less, 55° C. or less, 50° C. or less, 45° C. or less, 35° C. or less, or 25° C. or less).

The first digestate can be anaerobically incubated at a temperature ranging from any of the minimum values described above to any of the maximum values described above. For example, in some embodiments, the first digestate can be anaerobically incubated at a temperature of from 25° C. to 90° C., such as from 35° C. to 80° C., from 35° C. to 75° C., from 35° C. to 65° C., from 45° C. to 65° C., or from 55° C. to 60° C.

The first and second digestion reactions may be performed at the same temperatures or at different temperatures depending on the specific process parameters.

In some embodiments, the first digestate is contacted with a second inoculant prior to being anaerobically incubated. The second inoculant can include a mixed microbial community that is substantially the same as the inoculant of the alkaline digestion. In some embodiments, it is beneficial to contact the first digestate with a second inoculant comprising different microorganisms to increase the conversion of VFAs to a biogas. The second inoculant can include microorganisms chosen for the selective conversion of VFAs to a specific biogas product (e.g., methane). For example, the second inoculant can include mixed microbials enriched in methanogens and/or syntrophic acetate oxidizing bacteria (SAOB).

The method may produce a first biogas and a second biogas comprising methane. In some embodiments, the first biogas comprises at least 85% methane by volume, such as at least 89% methane by volume, at least 90% methane by volume, at least 95% methane by volume, at least 97% methane by volume, at least 99% methane by volume. The method may produce methane in volumetric amounts of at least 10 mL/g lignocellulosic biomass fed, at least 15 mL/g lignocellulosic biomass fed, at least 20 mL/g lignocellulosic biomass fed, at least 25 mL/g lignocellulosic biomass fed, at least 30 mL/g lignocellulosic biomass fed, or at least 40 mL/g lignocellulosic biomass fed. In addition, the method may yield a first biogas comprising at least 89% methane at a rate of at least 10 mL/g lignocellulosic biomass fed, at least 15 mL/g lignocellulosic biomass fed, at least 20 mL/g lignocellulosic biomass fed, at least 25 mL/g lignocellulosic biomass fed, at least 30 mL/g lignocellulosic biomass fed, or at least 40 mL/g lignocellulosic biomass fed. In other embodiments, the method may produce a first biogas comprising at least 90% methane at a rate of at least 10 mL/g lignocellulosic biomass fed, at least 15 mL/g lignocellulosic biomass fed, at least 20 mL/g lignocellulosic biomass fed, at least 25 mL/g lignocellulosic biomass fed, at least 30 mL/g lignocellulosic biomass fed, or at least 40 mL/g lignocellulosic biomass fed. Additional embodiments of the method produce a first biogas comprising at least 95% methane at a rate of at least 10 mL/g lignocellulosic biomass fed, at least 15 mL/g lignocellulosic biomass fed, at least 20 mL/g lignocellulosic biomass fed, at least 25 mL/g lignocellulosic biomass fed, at least 30 mL/g lignocellulosic biomass fed, or at least 40 mL/g lignocellulosic biomass fed.

In various embodiments, the method can produce a first biogas comprising a high purity of methane (e.g., at least 85% methane by volume, such as at least 89% methane by volume, at least 90% methane by volume, at least 95% methane by volume, at least 97% methane by volume, at least 99% methane by volume) in daily volumetric amounts of at least 1.0 mL/g lignocellulosic biomass fed per day, at least 1.5 mL/g lignocellulosic biomass fed per day, at least 2.0 mL/g lignocellulosic biomass fed per day, at least 2.5 mL/g lignocellulosic biomass fed per day, at least 3.0 mL/g lignocellulosic biomass fed per day, or at least 4.0 mL/g lignocellulosic biomass fed per day. The daily volumetric amount is defined as the average daily volumetric production of methane over the duration of the first retention time.

Compared to traditional anaerobic digesters fed with lignocellulosic biomass, the method disclosed herein produces a high purity biogas and a residual biogas stream with a high carbohydrate conversion. In certain embodiments, the carbohydrate conversion of the lignocellulosic biomass is at least 30% (e.g., at least 32%, at least 34%, at least 36%, at least 38%, at least 40%, at least 42%, at least 44%, at least 46%, at least 48%, at least 50%).

In various embodiments, the method processes the lignocellulosic feedstock at a daily carbohydrate conversion of at least 2.0% per day (e.g., at least 3.0% per day, at least 3.2% per day, at least 3.4% per day, at least 3.6% per day, at least 3.8% per day, at least 4.0% per day, at least 4.2% per day, at least 4.4% per day, at least 4.6% per day, at least 4.8% per day, at least 5.0% per day). The daily carbohydrate conversion refers to the average daily conversion of carbohydrates in the feedstock mixture the duration of the first retention time and second retention time as calculated using quantitative saccharification.

In some embodiments, a first digestate is produced after the feedstock mixture is anaerobically incubated for a first retention time. The first digestate may be a solid-liquid mixture comprising residual feedstock mixture, microbial biomass, and/or volatile fatty acids (VFAs). In some embodiments, the first digestate can include for example, VFAs comprising one or more selected from the group consisting of formate, acetate, propionate, butyrate, valerate, conjugates thereof, and combinations thereof. In certain embodiments, the VFAs may primarily comprise acetate. The VFA in the first digestate may be produced at a net production rate of at least 50 mg VFA/g lignocellulosic biomass fed, such as at least 75 mg VFA/g lignocellulosic biomass fed, at least 100 mg VFA/g lignocellulosic biomass fed, at least 125 mg VFA/g lignocellulosic biomass fed, at least 150 mg VFA/g lignocellulosic biomass fed, at least 175 mg VFA/g lignocellulosic biomass fed, at least 200 mg VFA/g lignocellulosic biomass fed, at least 400 mg VFA/g lignocellulosic biomass fed, or at least 500 mg VFA/g lignocellulosic biomass fed. In other embodiments, the VFA may be produced, from 50 to 500 mg VFA/g lignocellulose biomass fed, such as from 50 to 200 mg VFA/g lignocellulosic biomass fed, from 75 to 200 mg VFA/g lignocellulosic biomass fed, from 100 to 200 mg VFA/g lignocellulosic biomass fed, from 150 to 200 mg VFA/g lignocellulosic biomass fed, or from 175 to 200 mg VFA/g lignocellulosic biomass fed.

The second digester may convert some of the VFAs in the first digestate into a biogas comprising methane and carbon dioxide. VFA conversion is measured from the reduction in the concentration of VFAs present in the first digestate to the concentration of VFAs present in the second digestate. In some embodiments, the VFA conversion may be at least 40%, such as at least 45%, at least 50%, at least 55%, at least 60%, at least 65%, at least 70%, at least 75%, at least 80%, at least 85%, at least 90%, at least 95%, or substantially all.

In various embodiments, it is beneficial to cotreat the feedstock mixture and/or the digestate during the digestion process. The term “cotreatment” as used herein refers to a process for lowering the recalcitrance effects of the biomass by improving the cellulosic solubilization during the fermentation process. Unlike pretreatment, where degradation of a biomass occurs prior to a fermentation step, cotreatment can advantageously improve carbohydrate solubilization at a reduced energy demand, thereby making the process more economical and environmentally sustainable.

Cotreatment of a biomass (e.g., the feedstock mix and/or the first digestate) can be achieved by using, for example, mechanical treatment (e.g., milling), thermal treatment (e.g., hydrothermal heating with steam), chemical treatment (e.g., treatment with CaO), or enzymatic hydrolysis of the biomass. Cotreatment can occur in the digestion reaction vessel or elsewhere through recirculation of the biomass. In some embodiments, cotreatment of a biomass, such as cotreatment by mechanical milling, is performed continuously through the duration of the method (e.g., constant milling). In other embodiments, the biomass can be treated intermittently, such as by mechanical milling for one or more time periods during a fermentation stage (i.e., intermittent milling) and/or for a period in between stages in processes having multiple fermentation steps. In various embodiments, the methods include milling the feedstock mixture during the first retention time. In some embodiments, the methods include milling the first digestate during the second retention time in addition to or instead of the cotreatment of the feedstock mixture. In various embodiments, the feedstock mixture is milled intermittently for a period ranging from 0.5 minutes to 120 minutes, for example, from 0.5 minutes to 100 minutes, from 0.5 minutes to 80 minutes, from 0.5 minutes to 60 minutes, from 0.5 minutes to 40 minutes, from 0.5 minutes to 30 minutes, from 0.5 minutes to 20 minutes, from 0.5 minutes to 10 minutes, from 0.5 minutes to 5 minutes, from 1 minute to 120 minutes, from 5 minutes to 120 minutes, from 10 minutes to 120 minutes, from 20 minutes to 120 minutes, from 30 minutes to 120 minutes, or from 60 minutes to 120 minutes. Similarly, in some embodiments, the first digestate is milled intermittently for a period ranging from 0.5 minutes to 120 minutes, for example, from 0.5 minutes to 100 minutes, from 0.5 minutes to 80 minutes, from 0.5 minutes to 60 minutes, from 0.5 minutes to 40 minutes, from 0.5 minutes to 30 minutes, from 0.5 minutes to 20 minutes, from 0.5 minutes to 10 minutes, from 0.5 minutes to 5 minutes, from 1 minute to 120 minutes, from 5 minutes to 120 minutes, from 10 minutes to 120 minutes, from 20 minutes to 120 minutes, from 30 minutes to 120 minutes, or from 60 minutes to 120 minutes.

Mechanical cotreatment in the form of milling can effectuate an increase the conversion of cellulosic biomass into desired products. Mechanical cotreatment in the form of milling can increase degradation rates by exposing recalcitrant areas of cellulose to the mixed microbial community for digestion. The mechanical agitation can also enhance digestion by disrupting the biofilms on cellulosic particles to encourage new microbial colonization.

In various embodiments, the mechanical cotreatment includes milling of the reactor mixture using ball milling. Cotreatment via ball milling generally includes loading the bioreactor with a plurality of ball bearings (e.g., stainless-steel balls) which can subsequently be agitated to mechanically digest the reactor mixture (e.g., the feedstock mixture or first digestate). Another mechanical milling method utilizes a colloid mill to reduce lignocellulose recalcitrance. Colloid mills are generally configured with a rotating cone (typically rotating at high-speeds) inside a static cone with a small, adjustable gap between the rotor and the stator. These two parts have teeth and when rotated, the rotating head provides the motive force to pump a reactor mixture through where shear forces from contacting the teeth disrupt solid particles and cause a reduction in size.

Chemical cotreatment can involve the addition of a chemical cotreatment agents such as an oxidizing agent (e.g., hydrogen peroxide, peracetic acid) or other chemicals (e.g., acids and bases) that can disrupt the cellulosic structure by chemically exposing the lignocellulosic fibers for digestion. In some examples, a chemical cotreatment agent (e.g., an acid such as sulfuric acid, nitric acid or a base such as sodium hydroxide) can be added to the biomass for a cotreatment period (e.g., from 0.5 minutes to 120 minutes, for example, from 0.5 minutes to 100 minutes, from 0.5 minutes to 80 minutes, from 0.5 minutes to 60 minutes, from 0.5 minutes to 40 minutes, from 0.5 minutes to 30 minutes, from 0.5 minutes to 20 minutes, from 0.5 minutes to 10 minutes, from 0.5 minutes to 5 minutes, from 1 minute to 120 minutes, from 5 minutes to 120 minutes, from 10 minutes to 120 minutes, from 20 minutes to 120 minutes, from 30 minutes to 120 minutes, or from 60 minutes to 120 minutes). Following the cotreatment period, an amount of a soluble pH adjusting agent can be added to return the feedstock mixture to the alkaline pH or a neutral pH depending on the digestion stage. Preferably, the use of chemical cotreatment involves soluble chemical compounds that can maintain the desired process parameters while limiting the need for post-fermentation processing and separation. In some embodiments, the chemical cotreatment agent is chosen based on a reduced production of toxic and inhibitory compounds (e.g., phenolic compounds, furfural and hydroxylmethylfurfural) formed during the degradation of cellulosic material.

In some embodiments, it is beneficial to use a combination of cotreatment strategies to improve carbohydrate conversion of the lignocellulose biomass.

Some embodiments may further be benefitted from the addition of a nitrogen source to increase anaerobic digestion in either the first or second reactor. In some embodiments, the method may comprise adding a nitrogen source to the feedstock mixture. The nitrogen source may, for example, be any suitable nitrogen source, including but not limited to, ammonium salts, yeast extract, corn steep liquor (CSL), and other protein sources.

The second digestate may be recovered and processed according to any method known in the art, including but not limited to liquid-solid separation, material recycling, further biological, chemical, and/or physical treatment, storage, and/or utilization.

Methods of Forming VFAs from a Lignocellulosic Feedstock

Also disclosed herein are methods for producing volatile fatty acids (VFAs) from a lignocellulosic biomass. VFAs are a class of molecules that include straight and branched chain fatty acids and corresponding conjugates having carbon chain lengths from C2 to C6, including but not limited to acetic acid, propionic acid, butyric acid, isobutyric acid, 2-methyl butyric acid, valeric acid, isovaleric acid, and caproic acids. Longer chains of fatty acids (e.g., carbon chain lengths from C8 to C22) can be converted from VFAs by certain plant and animal products. VFAs are important in the subsequent synthesis of various chemical products, for example, alcohols, ketones, esters, olefins and aldehydes.

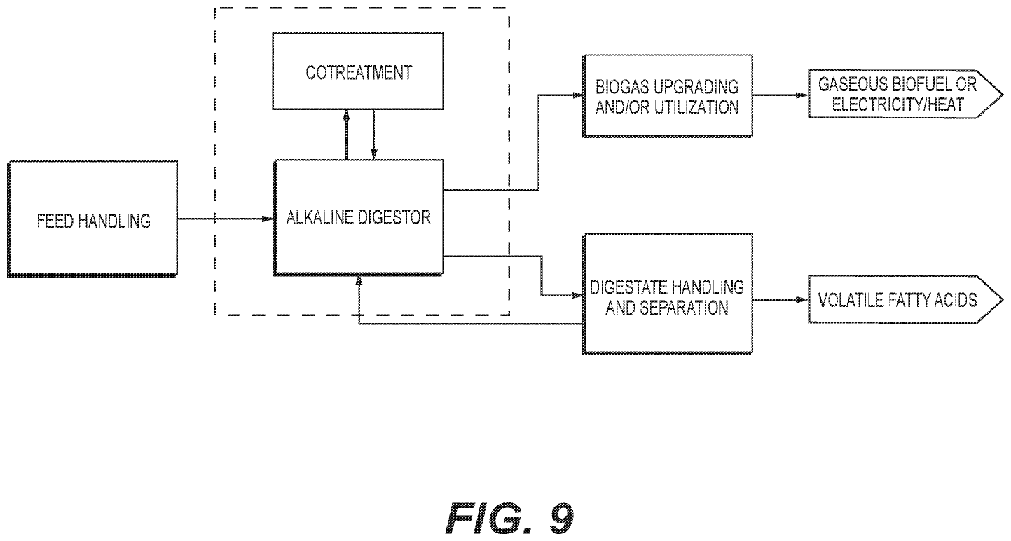

Various embodiments of the present method include inoculating a feedstock mixture including a lignocellulosic biomass with a mixed microbial community; contacting the feedstock mixture with effective amount of a pH adjusting agent to increase a pH of the feedstock mixture to an alkaline pH; and incubating the feedstock mixture anaerobically for a retention time at a thermophilic temperature of at least 45° C. thereby forming a digestate comprising a VFA. A representative block diagram showing one embodiment of a single-stage anaerobic digestor is shown in FIG. 9.

In some embodiments, the digestate can include VFAs comprising one or more selected from the group consisting of formate, acetate, propionate, butyrate, valerate, conjugates thereof, and combinations thereof. In certain embodiments, the VFAs primarily comprises acetate. The VFA in the digestate may be produced at a net production rate of at least 50 mg VFA/g lignocellulosic biomass fed, such as at least 75 mg VFA/g lignocellulosic biomass fed, at least 100 mg VFA/g lignocellulosic biomass fed, at least 125 mg VFA/g lignocellulosic biomass fed, at least 150 mg VFA/g lignocellulosic biomass fed, at least 175 mg VFA/g lignocellulosic biomass fed, at least 200 mg VFA/g lignocellulosic biomass fed, at least 400 mg VFA/g lignocellulosic biomass fed, or at least 500 mg VFA/g lignocellulosic biomass fed. In other embodiments, the VFA may be produced, from 50 to 500 mg VFA/g lignocellulose biomass fed, such as from 50 to 200 mg VFA/g lignocellulosic biomass fed, from 75 to 200 mg VFA/g lignocellulosic biomass fed, from 100 to 200 mg VFA/g lignocellulosic biomass fed, from 150 to 200 mg VFA/g lignocellulosic biomass fed, or from 175 to 200 mg VFA/g lignocellulosic biomass fed.

As presented above in the discussion of methods for forming biogas, the lignocellulosic biomass can be inoculated (e.g., by depositing, growing, or treating) with an inoculant. In some embodiments, the inoculant comprises a mixed microbial community. The mixed microbial community can comprise, for example, one or more methanogenic microorganisms. In certain embodiments, the mixed microbial community can comprise one or more types of lignocellulosic degrading microorganisms, including, for example, lignocellulosic degrading bacteria and lignocellulosic degrading fungi. In certain embodiments, the mixed microbial community can be obtained from one or more sources of the group consisting of bovine rumen fluid, bovine rumen solids, corn silage, compost, wetland sediment, and/or an existing anaerobic digester at wastewater treatment plant, farm, or other industrial facility. In various embodiments, the inoculant includes one or more types of fibrolytic bacteria including, for example, Fibrobacter succinogenes, Ruminococcus flavefaciens, Ruminococcus albus, Butyrivibrio fibrisolvens, Prevotella ruminicola, Eubacterium cellulosolvens, Eubacterium ruminantium, and combinations thereof, and/or one or more rumen fungi such as Piromyces, Neocallimastix, Orpinomyces, Ruminomyces, and combinations thereof.

In various embodiments, the inoculant (e.g., the mixed microbial community) includes one or more genetically modified microorganism(s) such as those disclosed in U.S. Pat. No. 10,662,456. As used herein, a “genetically modified microorganism” and the like refers to the direct human manipulation of a nucleic acid using modern DNA technology. For example, genetic manipulation can involve the introduction of exogenous nucleic acids into an organism or altering or modifying an endogenous nucleic acid sequence present in the organism. For example, a genetic modification can be insertion of a nucleotide sequence into the genome of a microorganism. A genetic modification can also be a deletion or disruption of a polynucleotide that encodes or regulates production of an endogenous or exogenous gene. A genetic modification can also result in the mutation of a nucleic acid or polypeptide sequence. For example, the inoculant can include a microorganism genetically modified to express or overexpress a polypeptide such as cellulase, endoglucanase, cellobiohydrolase, beta-glucosidase. In some embodiments, the inoculant includes one or more microorganisms that are engineered to be tolerant to environmental conditions of the bioreactor (e.g., pH, temperature, concentration of a toxin).

In some embodiments, the inoculant includes a genetically modified microorganism made to increase and/or decrease the cellular production of certain fermentation product(s) such as acetate, acetoin, acetone, acrylic, malate, fatty acid ethyl esters, isoprenoids, glycerol, ethylene glycol, ethylene, propylene, butylene, isobutylene, ethyl acetate, vinyl acetate, other acetates, 1,4-butanediol, 2,3-butanediol, butanol, isobutanol, sec-butanol, butyrate, isobutyrate, 2-OH-isobutryate, 3-OHbutyrate, ethanol, isopropanol, D-lactate, L-lactate, pyruvate, itaconate, levulinate, glucarate, glutarate, caprolactam, adipic acid, propanol, isopropanol, fusel alcohols, and 1,2-propanediol, 1,3-propanediol, formate, fumaric acid, propionic acid, succinic acid, valeric acid, maleic acid, and poly-hydroxybutyrate.

During incubation, the accumulation of VFAs can facilitate a drop in pH and/or lead to enzymatic inhibition of the microbes within the reactor. Thus, in some embodiments, it is advantageous to remove VFAs from the reactor during or subsequent anaerobic incubation. Separation of the VFAs from the reactant mixture can be accomplished by a variety of known methods, including, for example, gas stripping with absorption, adsorption, solvent extraction, electrodialysis, distillation, reverse osmosis (RO), nanofiltration (NF) and other membrane-based separation techniques. (Atasoy et al., 2018, Bioresource Tech. 268:773-786). In addition to batch removal of VFAs, where separation occurs following the fermentation process, the present methods also provide for continuous and semi-continuous in situ recovery of VFA products to reduce pH and inhibitory effects resulting from accumulation.

In some embodiments, VFAs can be allowed to accumulate to trigger the formation of higher order VFAs (e.g., medium-chain carboxylic acids and long-chain carboxylic acids) and isoforms thereof, which can be separated as a commodity chemical. These higher order VFAs can include, for example, one or more of caproate acid, heptanoate acid, caprylate acid, nonanoic acid, lauric acid, lauroleic acid, myristic acid, myristoleic acid, pentadecanoic acid, palmitic acid, palmitoleic acid, margaric acid, stearic acid, dihydroxystearic acid, oleic acid, ricinoleic acid, elaidic acid, linoleic acid, alpha-linolenic acid, dihomogamma-linolenic acid, eleostearic acid, licanic acid, arachidonic acid, arachidic acid, eicosenoic acid, eicosapentaenoic acid, behenic acid, erucic acid, docosahexaenoic acid, lignoceric acid, and conjugates thereof.

### Systems for the Conversion of a Lignocellulosic Feedstock

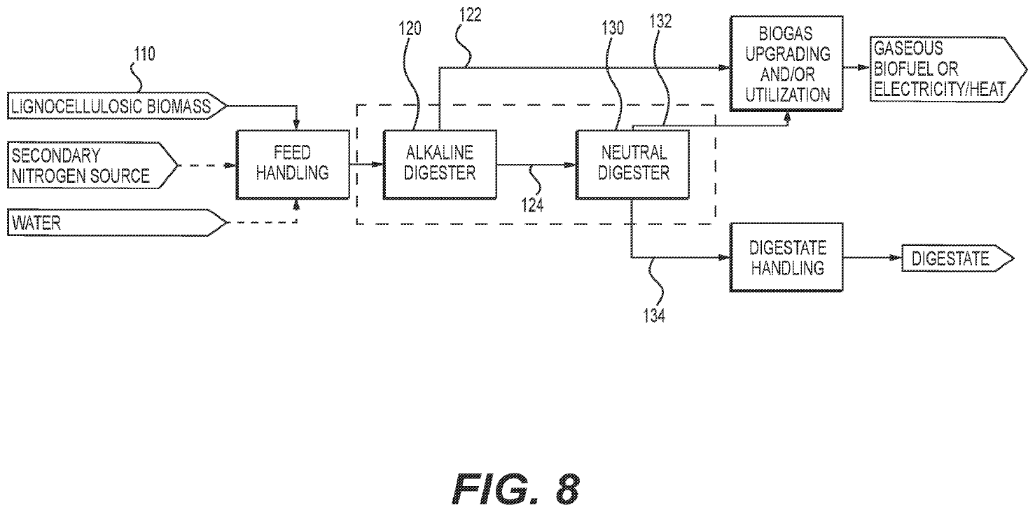

Disclosed herein are systems for the conversion of a lignocellulosic feedstock 110 to a biogas. As shown in FIG. 8, the systems include a first reactor 120; wherein the first reactor 120 is configured to receive the lignocellulosic feedstock 110 and a mixed microbial community; and wherein the first reactor 120 operates at an alkaline pH and a thermophilic temperature to anaerobically digest the lignocellulosic feedstock 110 for a first retention time thereby producing a first biogas 122 and a first digestate 124 comprising volatile fatty acids; and a second reactor 130 configured to receive the first digestate 124; wherein the second reactor 130 operates at a substantially neutral pH to anaerobically digest the first digestate 124 for a second retention time thereby producing a second biogas 132 and a second digestate 134.

The first reactor operates to produce a high purity biogas product substantially comprising methane. The first biogas may comprise at least 85% methane by volume (e.g., at least 89% methane by volume, at least 90% methane by volume, at least 95% methane by volume, at least 97% methane by volume, at least 99% methane by volume). The first biogas may be sequestered from the reactor and collected where it may be utilized as a renewable fuel source. Further refinement of the biogas product may be implemented according to its desired use.

The second reactor can also operate to produce a second biogas product comprising methane and carbon dioxide. The second biogas may comprise at from 1% to 90% methane by volume (e.g., from 1% to 80% by volume, from 1% to 70% by volume, from 1% to 60% by volume, from 1% to 50% by volume, from 1% to 40% by volume, from 1% to 30% by volume, from 1% to 20% by volume, from 1% to 10% by volume, from 10% to 50% by volume, from 20% to 50% by volume, from 30% to 50% by volume, from 40% to 50% by volume, from 50% to 90% by volume, from 60% to 90% by volume, from 70% to 90% by volume, from 80% to 90% by volume). The second biogas may be sequestered from the reactor and collected where it may be utilized as a renewable fuel source. Further refinement of the biogas product may be implemented according to its desired use.

Various embodiments of the present system include a cotreatment vessel wherein the lignocellulose biomass can be treated during anaerobic digestion. In some examples, the cotreatment vessel comprises one or both of the first and second reactors. The cotreatment vessel can also define a separate location from the first and second reactors where the lignocellulose feedstock and/or first digestate are transferred for processing using one or more of the cotreatment techniques discussed above. In various embodiments, the first reactor and/or the second reactor includes a milling device disposed therewithin. As discussed above, the milling device is used to cotreat the lignocellulose biomass during the digestion process. The cotreatment can lower the recalcitrance effects of the biomass by improving the cellulosic solubilization during the fermentation process. In some embodiments, cotreatment of a biomass is performed continuously through the duration of the method (i.e., constant milling). In other embodiments, it is beneficial to cotreat the biomass intermittently, such as mechanical milling for one or more time periods during a fermentation stage (i.e., intermittent milling) and/or for a period in between stages in processes having multiple fermentation steps.

As discussed above, mechanical cotreatment in the form of milling can increase degradation rates by exposing recalcitrant areas of cellulose to the mixed microbial community for digestion. The mechanical agitation can also enhance digestion by disrupting the biofilms on cellulosic particles to encourage new microbial colonization.

In various embodiments, the mechanical cotreatment includes milling of the reactor mixture using ball milling. Cotreatment via ball milling generally includes loading the bioreactor with a plurality of ball bearings (e.g., stainless-steel balls) which can subsequently be agitated to mechanically digest the reactor mixture (e.g., the feedstock mixture or first digestate).

Colloid mills are generally configured with a rotating cone (typically rotation at high-speeds) inside a static cone with a small, adjustable gap between the rotor and the stator. These two parts have teeth and when rotated, the rotating head provides the motive force to pump a reactor mixture through where shear forces from contacting the teeth disrupt solid particles and cause a reduction in size.

### EXAMPLES

The following examples are put forth so as to provide those of ordinary skill in the art with a complete disclosure and description of how the compounds, compositions, articles, devices, and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by volume, the temperature is in degrees C. or is at ambient temperature, and pressure is at or near atmospheric.

### Example 1

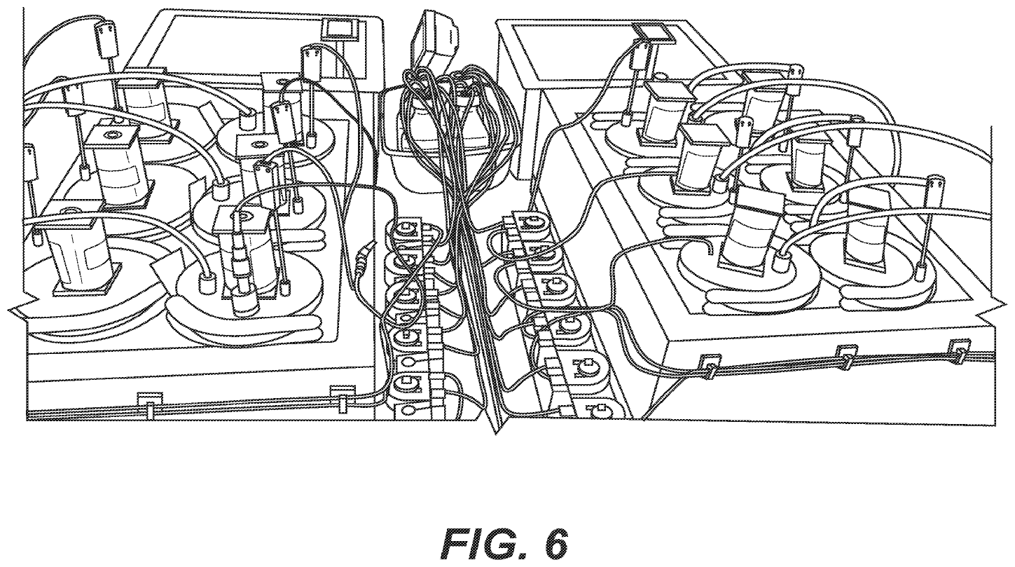

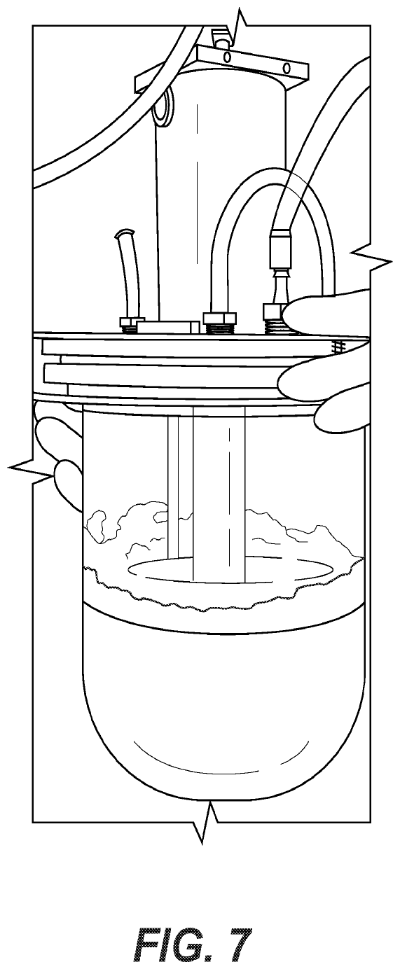

Described herein is a triplicate set of lab-scale well-mixed liquid-state reactors fed semi-continuously on unpretreated senescent switchgrass. Example lab-scale reactor vessels are shown in FIG. 6 and FIG. 7.

### Method

A minimal medium was mixed with the switchgrass to create a slurry as well as provide a nitrogen source, trace minerals, and nutrients (Table 1). Reactors were operated at 55° C., pH 8.5, and with a retention time of 10 d. The organic loading rate was 2.0 g switchgrass volatile solids (VSsg)/L/day with feeding occurring once every 24 h.

TABLE 1
Formulation of anaerobic minimal medium, adapted from Angelidaki et al. (2009).
Final concentrations are given in mg/L, except where otherwise indicated.

| Compound | Concentration | Compound | Concentration | |
|---|---|---|---|---|
| NH₄Cl | 1000 | FeCl₂ 4H2O | 2 | |
| NaCl | 100 | ZnCl₂ | 0.05 | |
| MgCl₂ 6H2O | 100 | Na₂SeO₃ 5H2O | 0.1 | |
| CaCl₂ 2H2O | 50 | Na₂WO₄ 2H2O | 0.1 | |
| KH₂PO₄ | 400 | Biotin | 2 | μg/L |
| HCl | 0.0012 | Folic acid | 2 | μg/L |
| H₃BO₃ | 0.05 | Pyridoxine hydrochloride | 10 | μg/L |
| Nitrilotriacetic acid | 1 | Riboflavin | 5 | μg/L |
| CuCl₂ 2H2O | 0.038 | Thiamine hydrochloride | 5 | μg/L |
| MnCl₂ 4H2O | 0.05 | Cyanocobalamin | 0.1 | μg/L |
| (NH₄)₆Mo₇O₂₄ 4H2O | 0.05 | Nicotinic acid | 5 | μg/L |
| AlCl₃ | 0.05 | p-Aminobenzoic acid | 5 | μg/L |
| CoCl₂ 6H2O | 0.05 | Lipoic acid | 5 | μg/L |
| NiCl₂ 6H2O | 0.092 | Pantothenic acid | 5 | μg/L |

The system was inoculated at a feed to inoculum ratio of 2:1 on a volatile solids (VS) basis. Inoculum was from six sources: bovine rumen fluid, bovine rumen solids, corn silage, compost, wetland sediment, and wastewater treatment plant anaerobic sludge. After inoculation, the well-mixed reactor was held at the specified operating temperature and pH for a five-day batch incubation prior to beginning the semi-continuous feeding regime described above.

### Results

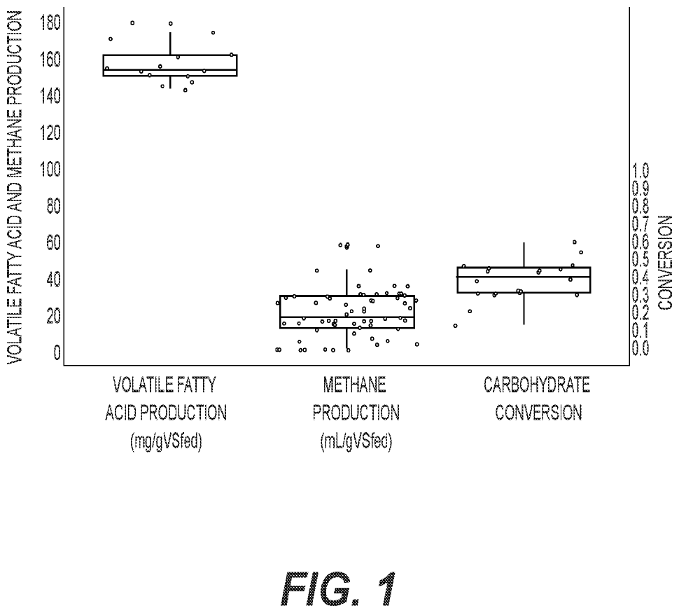

After three retention times the system was determined to be at process steady state, based on gas production, volatile fatty acid production, and carbohydrate conversion. Median steady state product formation was 152.6 mg VFA/g VS_{sg} fed with 97 wt % acetic acid, 17.6 mL methane/g VS_{sg} fed at 96 vol % methane, and carbohydrate conversion of 40.6%, as measured by quantitative saccharification. Values are from samples taken across two retention times and three biological replicates. Complete data sets are shown in FIG. 1.

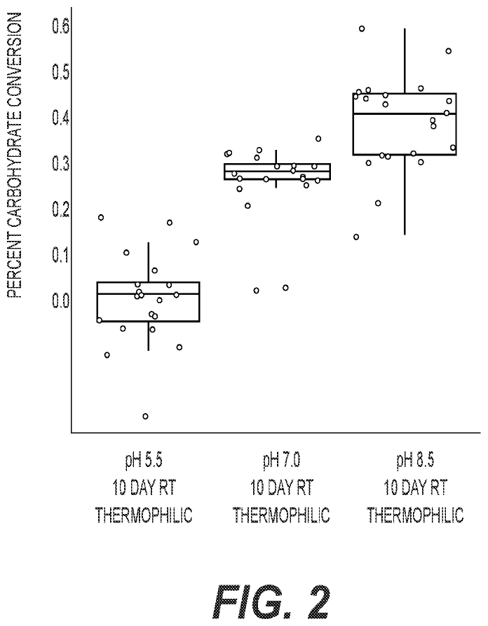

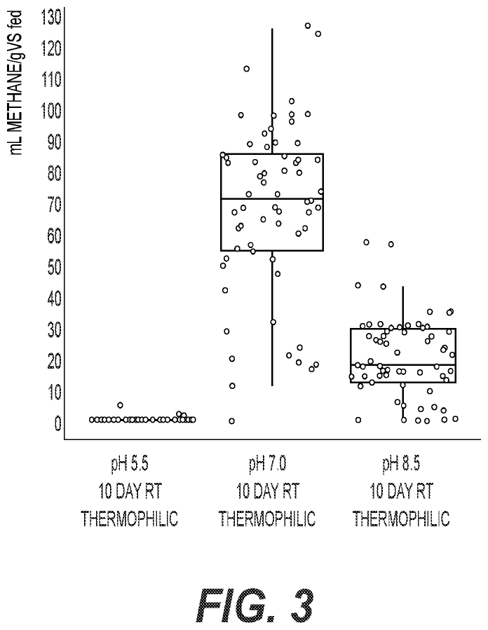

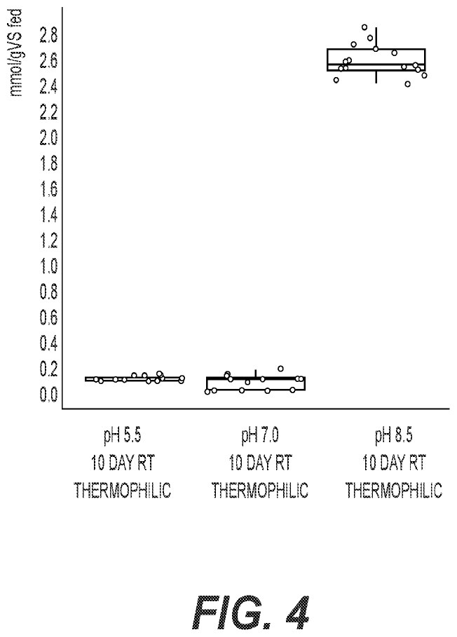

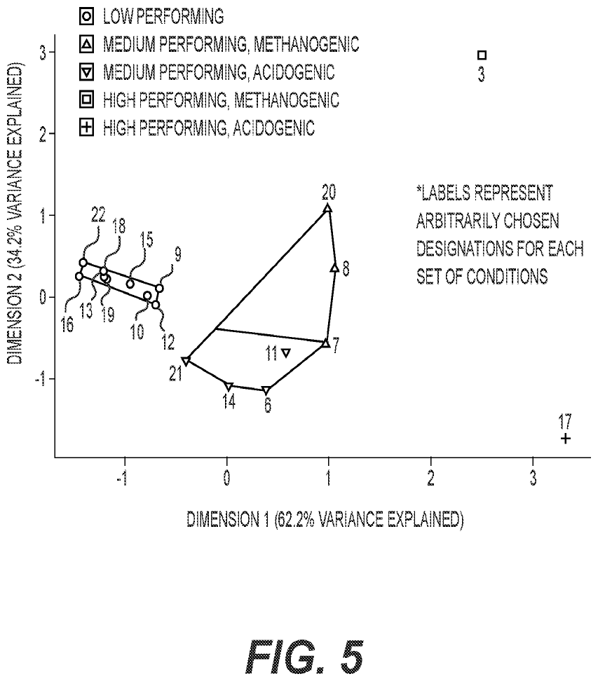

In contrast, the experiments described above were also performed with acidic (pH 5.5) and neutral (pH 7.0) conditions maintaining the same temperature and retention time. Median steady state product formation under acidic pH was 5.5 mg VFA/g VS_{sg} fed with only acetic acid detected, no measurable biogas production, and 1.3% carbohydrate conversion. The neutral pH condition performed better than acidic pH with product formation of 5.5 mg VFA/g VS_{sg} fed as acetic acid, 71.5 mL methane/g VS_{sg} fed at 73 vol % methane, and 27.8% carbohydrate conversion. Compared to acidic and neutral conditions, alkaline digestion increased carbohydrate conversion by 39.3% and 12.8%, respectively. Methane concentration also increased 23 vol %. The VFAs generated during this alkaline digestion can be further converted in a subsequent anaerobic digestion or separated for use in the processing of various chemical products, such as alcohols, ketones, aldehydes, and olefins. A comparison of the effects of pH and temperature on anaerobic digestion production and summary of the process conditions can be found in FIG. 2-FIG. 5 and in Table 2-Table 4.

TABLE 2
Comparison of alkalinity and temperature on product formation.

| Retention | | | | Biogas | Methane | Volatile Fatty Acid | Acetate Fraction of |
|---|---|---|---|---|---|---|---|
| Time | Temperature | | Carbohydrate | Production | Concentration | Net Production | Volatile Fatty Acid |
| (days) | (° C.) | pH | Conversion | (mL/gVS fed) | (vol %) | (mmol/gVS fed) | (mol %) |
| 3.33 | 37 | 5.5 | −3.2% | 0.00 | | 0.14 | 100% |
| 10 | 37 | 5.5 | −0.9% | 0.00 | | 0.08 | 0% |
| 5 | 55 | 5.5 | 0.7% | 0.00 | | 0.15 | 100% |
| 5 | 37 | 5.5 | 0.9% | 0.00 | | 0.20 | 85% |
| 10 | 55 | 5.5 | 1.3% | 0.00 | | 0.09 | 100% |
| 3.33 | 55 | 5.5 | 4.1% | 0.00 | | 0.23 | 100% |
| 5 | 37 | 7.0 | 5.5% | 0.00 | | 0.36 | 100% |
| 3.33 | 37 | 8.5 | 5.7% | 0.00 | | 0.59 | 77% |
| 3.33 | 37 | 7.0 | 8.3% | 0 | | 0.28 | 97% |
| 10 | 37 | 8.5 | 4.7% | 3.22 | 99.1% | 1.34 | 87% |
| 5 | 37 | 8.5 | 7.7% | 0.00 | | 1.46 | 91% |
| 5 | 55 | 8.5 | 10.3% | 3.12 | 94.2% | 1.47 | 100% |
| 3.33 | 55 | 8.5 | 17.0% | 4.83 | 97.5% | 1.21 | 93% |
| 10 | 37 | 7.0 | 21.2% | 35.18 | 80.2% | 0.50 | 68% |
| 3.33 | 55 | 7.0 | 21.4% | 14.75 | 55.4% | 1.18 | 98% |
| 5 | 55 | 7.0 | 22.9% | 26.55 | 76.3% | 0.78 | 89% |
| 10 | 55 | 7.0 | 27.8% | 101.68 | 67.9% | 0.09 | 100% |
| 10 | 55 | 8.5 | 40.0% | 18.05 | 97.7% | 2.54 | 97% |

TABLE 3
pH control system precision.
pH

| | | Standard | 95% confidence |
|---|---|---|---|
| Condition | Mean | Deviation | Interval |
| 10 day RT, | 5.47 | ±0.11 | ±0.0011 |
| Thermophilic, pH 5.5 | | | |
| 10 day RT, | 7.00 | ±0.08 | ±0.0005 |
| Thermophilic, pH 7.0 | | | |
| 10 day RT, | 8.48 | ±0.21 | ±0.0021 |
| Thermophilic, pH 8.5 | | | |

TABLE 4
Temperature control system precision.
Temperature

| | | Standard | 95% confidence |
|---|---|---|---|
| Condition | Mean | Deviation | Interval |
| 10 day RT, | 56.5° C. | ±0.7 | ±0.007 |
| Thermophilic, pH 5.5 | | | |
| 10 day RT, | 56.4° C. | ±0.7 | ±0.005 |
| Thermophilic, pH 7.0 | | | |
| 10 day RT, | 56.0° C. | ±0.5 | ±0.005 |
| Thermophilic, pH 8.5 | | | |

After the first alkaline reactor, the digestate was recovered and processed in a sequential reactor operating at a substantially neutral pH. This second stage promotes the conversion of the suite of VFAs produced in the first alkaline digester to further produce methane and carbon dioxide. The second digester produces a second biogas and a second digestate. The gaseous stream leaving the second reactor may be combusted or further refined to yield renewable natural gas. A block diagram showing this setup can be seen in FIG. 8.

### Example 2

Described herein is a duplicate set of lab-scale well-mixed liquid-state reactors fed semi-continuously on unpretreated senescent switchgrass. Example lab-scale reactor vessels are shown in FIG. 6 and FIG. 7.

### Materials and Methods

A minimal medium was mixed with the switchgrass to create a slurry as well as provide a nitrogen source, trace minerals, and nutrients (Table 1). Reactors were operated at 55° C., a retention time of 10 d, and six pH conditions ranging from pH 7.3 to pH 10.3 at 0.6 pH unit increments. The organic loading rate was 2.0 g switchgrass volatile solids (VSsg)/L/day with feeding occurring once every 24 h. The feed included the same formulation of anaerobic minimal medium, adapted from Angelidaki et al. (2009) previously described.

The system was inoculated at a feed to inoculum ratio of 2:1 on a volatile solids (VS) basis. Inoculum was from six sources: bovine rumen fluid, bovine rumen solids, corn silage, compost, wetland sediment, and wastewater treatment plant anaerobic sludge. After inoculation, the well-mixed reactor was held at the specified operating temperature and pH for a five-day batch incubation prior to beginning the semi-continuous feeding regime described above.

Results shown in FIG. 8 and FIG. 9 are from samples withdrawn from each reactor at each of four retention times: 3.0, 5.0, 6.2, and 7.0, and with duplicate reactors there were 8 measurements for each pH condition. As in the previous example, the primary carboxylic acid produced was acetic acid, with small amounts of formic, propanoic, and butyric acid also measured as shown in FIG. 8. Mean values and standard deviations of the millimoles of total VFAs per g VS fed are presented in FIG. 9. VFA production was highest at pH 8.5 and pH 9.1, with statistically higher conversion rates observed in this pH range than at conditions below pH 7.9 or above pH 9.7.

TABLE 5
Median conversion and product profile for each experimental condition calculated with combined data from all biological
replicates and samples collected in the fourth and fifth retention times. Conditions are defined by Retention Time
(RT, including 3.3, 5, and 10 days), Temperature (mesophilic = M, thermophilic = T) and pH (5.5, 7.0,
and 8.5). All conditions were run in triplicate. Red boxes highlight thermophilic alkaline pH conditions.

| | | | | Biogas | | Volatile Fatty Acid | | | |
|---|---|---|---|---|---|---|---|---|---|
| | | | Carbohydrate | Production | | Net Production | | Alpha | |
| | | Condition Set | Conversion | (mL/ [symbol] ) | | (mmol/ [symbol] ) | | Diversity | Dominant [symbol] |
| Performance group | | (RT_Temp_pH) | (%) | (vol % CH [symbol] ) | | (mol % Acetate) | | (observed [symbol] ) | ( [symbol] Abundance, %) |
| Low | | 3.3_M_5.5 | −3.2 [symbol] | 0 | | 0.8 | (100) | 873 | Bacteroidota (42.9); |
| | | | | | | | | | Firmicutes (27.4); |
| | | | | | | | | | Proteobacteria (36.5) |
| | | 10_M_5.5 | −0.9 [symbol] | 0 | | 0.2 | (0) | 520 | Bacteroidota (52.9); |
| | | | | | | | | | Firmicutes (34.8); |
| | | | | | | | | | Halobacterota (3.9) |
| | | 5_T_5.5 | 0.7 | 0 | | 0.6 | (100) | 262 | Firmicutes (28.2); |
| | | | | | | | | | Thermotogota (27.6); |
| | | | | | | | | | Coprothermobacterota (22.9) |
| | | 5_M_5.5 | 0.9 | 0 | | 0.8 | (85) | 521 | Bacteroidota (56.3); |
| | | | | | | | | | Firmicutes (28.0); |
| | | | | | | | | | Actinobacteria (3.7) |
| | | 10_T_5.5 | 1.3 | 0 | | 0.2 | (100) | 420 | Coprothermobacterota (41.7); |
| | | | | | | | | | Firmicutes (42.9); |
| | | | | | | | | | Bacteroidota (10.0) |
| | | 3.3_T_ 5.5 | 4.3 | 0 | | 1.4 | (100) | 383 | Thermotogota (44.3); |
| | | | | | | | | | Firmicutes (25.7); |
| | | | | | | | | | Spirochaetota (18.9) |
| | | 5_M_7 | 5.5 | 0 | | 1.4 | (100) | 607 | Bacteroidota (56.6); |
| | | | | | | | | | Firmicutes (27.1); |
| | | | | | | | | | Fibrobacterota (4.2) |
| | | 3.3_M_8.5 | 5.7 | 0 | | 3.6 | (77) | 647 | Firmicutes (48.6); |
| | | | | | | | | | Bacteroidota (43.0); |
| | | | | | | | | | Actinobacteriota (2.9) |
| | | 3.3_M_7 | 8.3 | 0 | | 1.7 | (97) | 633 | Bacteroidota (52.1); |
| | | | | | | | | | Firmicutes (27.6); |
| | | | | | | | | | Fibrobacterota (5.1) |
| Medium | Favoring | 10_M_8.5 | 4.7 | 6.4 | (99.1) | 2.7 | (87) | 650 | Firmicutes (53.8); |
| | Acidogenesis | | | | | | | | Bacteroidota (30.4); |
| | | | | | | | | | Halobacterota (4.5) |
| | | 5_M_8.5 | 7.7 | 0 | | 5.8 | (91) | [symbol] 43 | Bacteroidota (46.9); |
| | | | | | | | | | Firmicutes (41.5); |
| | | | | | | | | | Proteobacteria (4.7) |
| | | 5_T_8.5 | 10.3 | 32.5 | (94.2) | 5.9 | (100) | 332 | Firmicutes (96.2); |
| | | | | | | | | | Euryarchaeota (2.3) [symbol] |
| | | 3.3_T_8.5 | 17 | 29.3 | (97.5) | 7.4 | (93) | 323 | Firmicutes (94.0); |
| | | | | | | | | | Euryarchaeota (3.8) [symbol] |
| | Favoring | 10_M_7 | 21.2 | 70.4 | (80.2) | 1.0 | (68) | 655 | Bacteroidota (42.8); |
| | Methanogenesis | | | | | | | | Firmicutes (34.0); |
| | | | | | | | | | Halobacterota (8.1) |
| | | 3.3_T_7 | 21.4 | 89.4 | (55.4) | 7.1 | (98) | 271 | Firmicutes (76.0); |
| | | | | | | | | | Bacteroidota (13.7); |
| | | | | | | | | | Halobacterota (3.6) |
| | | 5_T_7 | 22.0 | 106.2 | (76.3) | 3.3 | (80) | 4 [symbol] 3 | Firmicutes (73.1); |
| | | | | | | | | | Halobacterota (8.5); |
| | | | | | | | | | Bacteroidota (7.8) |
| High | Favoring | 10_T_7 | 27.8 | 203.4 | (67.9) | 0.2 | (100) | 350 | Firmicutes (57.8); |
| | Methanogenesis | | | | | | | | Halobacterota (15.7); |
| | | | | | | | | | Acidobacteriota (5.8) |
| High | Favoring | 10_T_8.5 | 40.6 | 36.1 | (97.7) | 5.3 | (97) | 466 | Firmicutes (90.2); |
| | Acidogenesis | | | | | | | | Bacteroidota (4.0); |
| | | | | | | | | | Euryarchaeota (2.2) [symbol] |

[symbol]

Belonging to the domain of Bacteria, unless otherwise specified; [symbol]
indicates domain Archaea [symbol]
Negative conversion is likely due to a prior acc [symbol] ation of biomass in the reactor thus raising the eff [symbol] ent carbohydrate concentration slightly above the feed in the fourth as a fifth retention times. [symbol]
indicates data missing or illegible when filed

### REFERENCES

Angelidaki, I., Alves, M., Bolzonella, D., Borzacconi, L., Campos, J. L., Guwy, A. J., et al. (2009) Defining the biomethane potential (BMP) of solid organic wastes and energy crops: A proposed protocol for batch assays. Water Sci Technol 59:927-934.
Beccari, M., Bonemazzi, F., Majone, M., and Riccardi, C. (1996) Interaction between acidogenesis and methanogenesis in the anaerobic treatment of olive oil mill effluent. Water Res 30:183-189.
Frigon, J. C., Mehta, P., and Guiot, S. R. (2012) Impact of mechanical, chemical and enzymatic pre-treatments on the methane yield from the anaerobic digestion of switchgrass. Biomass and Bioenergy 36:1-11.
Hendriks, A. T. W. M. and Zeeman, G. (2009) Pretreatments to enhance the digestibility of lignocellulosic biomass. Bioresour Technol 100:10-18.
Linville, J. L., Shen, Y., Ignacio-de Leon, P. A., Schoene, R. P., and Urgun-Demirtas, M. (2017) In-situ biogas upgrading during anaerobic digestion of food waste amended with walnut shell biochar at bench scale. Waste Manag Res 35:669-679.
Nolla-Ardevol, V., Strous, M., and Tegetmeyer, H. E. (2015) Anaerobic digestion of the microalga Spirulina at extreme alkaline conditions: Biogas production, metagenome and metatranscriptome. Front Microbiol 6:1-21.
Paul, S. and Dutta, A. (2018) Challenges and opportunities of lignocellulosic biomass for anaerobic digestion. Resour Conserv Recycl 130:164-174.
Shen, Y., Forrester, S., Koval, J., and Urgun-Demirtas, M. (2017) Yearlong semi-continuous operation of thermophilic two-stage anaerobic digesters amended with biochar for enhanced biomethane production. J Clean Prod 167:863-874.
Shen, Y., Linville, J. L., Ignacio-de Leon, P. A. A., Schoene, R. P., and Urgun-Demirtas, M. (2016) Towards a sustainable paradigm of waste-to-energy process: Enhanced anaerobic digestion of sludge with woody biochar. J Clean Prod 135:1054-1064.
Shen, Y., Linville, J. L., Urgun-Demirtas, M., Schoene, R. P., and Snyder, S. W. (2015) Producing pipeline-quality biomethane via anaerobic digestion of sludge amended with corn stover biochar with in-situ CO2 removal. Appl Energy 158:300-309.
Sousa, J. A. B., Sorokin, D. Y., Bijmans, M. F. M., Plugge, C. M., and Stams, A. J. M. (2015) Ecology and application of haloalkaliphilic anaerobic microbial communities. Appl Microbiol Biotechnol 99:9331-9336.
Jung, J., Lee, S., Shin, P., and Chung, Y. (2000) Effect of pH on Phase Separated Anaerobic Digestion. 456-459.
Lidner, J., Zielonka, S., Oeschner, H., and Lemmer, A. (2015) Effect of different pH-values on process parameters in two-phase anaerobic digestion of high solids substrates. Environ Technol 36:198-207.
Shen, Y., Forrester, S., Koval, J., and Urgun-Demirtas, M. (2017) Yearlong semi-continuous operation of thermophilic two-stage anaerobic digesters amended with biochar for enhanced biomethane production. J Clean Prod 167:863-874.

1. A system for the conversion of a lignocellulosic feedstock to a biogas, the system comprising:
a first reactor;
wherein the first reactor is configured to receive the lignocellulosic feedstock and a mixed microbial community; and
wherein the first reactor operates at an alkaline pH and a thermophilic temperature to anaerobically digest the lignocellulosic feedstock for a first retention time thereby producing a first biogas and a first digestate comprising volatile fatty acids; and
a second reactor configured to receive the first digestate;
wherein the second reactor operates at a substantially neutral pH to anaerobically digest the first digestate for a second retention time thereby producing a second biogas and a second digestate.

2. The system of claim 1, wherein the first biogas comprises at least 85% methane by volume.

3. The system of claim 1, wherein the alkaline pH is obtained by the addition of a first pH adjusting agent.

4. The system of claim 3, wherein the first pH adjusting agent comprises one or more selected from the group consisting of sodium bicarbonate, potassium bicarbonate, sodium carbonate, potassium carbonate, sodium hydroxide, potassium hydroxide, or any combination thereof.

5. The system of claim 1, wherein the lignocellulosic biomass comprises switchgrass.

6. (canceled)

7. (canceled)

8. (canceled)

9. The system of claim 1, wherein the alkaline pH is from 8.0 to 10.0.

10. The system of claim 1, wherein the thermophilic temperature is at least 45° C.

11. (canceled)

12. The system of claim 1, wherein the mixed microbial community is obtained from one or more of the group consisting of bovine rumen fluid, bovine rumen solids, corn silage, compost, wetland sediment, and/or an existing anaerobic digester at wastewater treatment plant, farm, or other industrial facility.

13. The system of claim 1, wherein the first digestate comprises volatile fatty acids and wherein the volatile fatty acid net production is at least 50 mg VFA/g lignocellulosic biomass fed.

14. The system of claim 13, wherein the VFAs primarily comprise acetate.

15. (canceled)

16. The system of claim 1, wherein the substantially neutral pH is obtained by the addition of a second pH adjusting agent.

17. The system of claim 16, wherein the pH adjusting agent comprises one or more selected from the group consisting of acetic acid, citric acid, hydrochloric acid, sulfuric acid, nitric acid, oxalic acid, sulfurous acid, carbonic acid, phosphoric acid, tartaric acid, boric acid, formic acid, or any combination thereof.

18. (canceled)

19. The system of claim 1, wherein the first reactor and/or the second reactor comprises a milling device disposed therewithin, wherein, during anaerobic digestion, the milling device is configured to deliver mechanical agitation to a mixture within the first reactor and/or the second reactor.

20. (canceled)

21. A method for forming a high purity biogas and a residual biogas from a lignocellulosic biomass, the method comprising the steps of:
a. inoculating a feedstock mixture comprising the lignocellulosic biomass with a mixed microbial community;
b. contacting the feedstock mixture with effective amount of a first pH adjusting agent to increase a pH of the feedstock mixture to an alkaline pH;
c. incubating the feedstock mixture anaerobically for a first retention time at a thermophilic temperature of at least 45° C. thereby forming a first biogas and a first digestate;
d. collecting the first biogas;
e. contacting the first digestate with effective amount of a second pH adjusting agent to decrease a pH of the first digestate to a substantially neutral pH;
f. incubating the first digestate anaerobically for a second retention time thereby forming a second biogas; and
g. collecting the second biogas.

22. The method of claim 21, wherein step (c) is performed in a first reactor and step (f) is performed in a second reactor, and wherein the method further comprises transferring the first digestate from the first reactor to the second reactor.

23. The method of claim 21, wherein the first biogas comprises 85% methane by volume.

24. (canceled)

25. (canceled)

26. (canceled)

27. (canceled)

28. (canceled)

29. The method of claim 21, wherein the first digestate comprises volatile fatty acids and wherein the volatile fatty acid net production is at least 50 mg VFA/g lignocellulosic biomass fed.

30. The method of claim 21, wherein the total carbohydrate conversion of the lignocellulosic biomass is greater than 30%.

31. (canceled)

32. (canceled)

33. (canceled)

34. (canceled)

35. The method of claim 21, wherein the alkaline pH is from 8.0 to 10.0.

36. (canceled)

37. The method of claim 21, further comprising milling the feedstock mixture during the first retention time.

38. (canceled)

39. (canceled)

40. A method for forming a volatile fatty acid (VFA) from a lignocellulosic biomass, the method comprising:
a. inoculating a feedstock mixture comprising the lignocellulosic biomass with a mixed microbial community;
b. contacting the feedstock mixture with effective amount of a pH adjusting agent to increase a pH of the feedstock mixture to an alkaline pH; and
c. incubating the feedstock mixture anaerobically for a retention time at a thermophilic temperature of at least 45° C. thereby forming a digestate comprising a VFA.

41. (canceled)

42. (canceled)

43. (canceled)

44. (canceled)

45. (canceled)
